(19) United States
(12) Patent Application Publication
(10) Pub. No.: US 2017/0007790 A1
(43) Pub. Date: Jan. 12, 2017

(54) SYSTEM AND METHOD FOR PURGING MOISTURE FROM THE INFLATION APPARATUS ON AN ARTIFICIAL AIRWAY

(71) Applicant: Shawn Walker, Andover, MA (US)

(72) Inventor: Shawn Walker, Andover, MA (US)

(21) Appl. No.: 15/196,744

(22) Filed: Jun. 29, 2016

Related U.S. Application Data
(60) Provisional application No. 62/188,885, filed on Jul. 6, 2015.

(51) Int. Cl. A61M 16/04; A61M 16/08; A61M 16/00

(52) U.S. Cl. CPC A61M 16/044; A61M 16/0051; A61M 16/0808; A61M 2205/3331; A61M 2205/502; A61M 2205/581; A61M 2205/583

(57) ABSTRACT

A system and method for periodically providing air pressure through an air line coupled to an air cuff in order to purge unwanted moisture from the air line while maintaining pressure in the air cuff. An air pressure measurement device is coupled to the air line by means of pneumatic circuit and determines whether the air pressure in the air line and coupled air cuff is equal to, greater than or less than a preprogrammed air pressure value. A pressure regulator receives the air measurement signal and is configured to control an air pressure device to increase or decrease the air pressure in the air line based upon the measured air pressure. A purge regulator controls the air pressure device to periodically cause the air pressure device to increase the air pressure in the air line to a predetermined level for a predetermined period of time at predetermined intervals in order to rid the air line of any accumulated moisture.

### CROSS-REFERENCE TO RELATED APPLICATIONS

This application claims priority from U.S. Provisional Patent Application No. 62/188,885 titled “SYSTEM AND METHOD FOR PURGING AN AIR CUFF ON AN ARTIFICIAL AIRWAY” filed on Jul. 6, 2015 and which is incorporated fully herein by reference.

### BACKGROUND

An endotracheal tube is one example of an artificial airway and is a term that describes a breathing tube that is inserted through the windpipe or trachea. It is commonly called an ETT or ET tube. Patients may need an endotracheal tube for one of several reasons. An endotracheal tube is needed to mechanically ventilate a patient (or breathe for them by a machine). Each breath is pushed into the endotracheal tube and into the lung.

An endotracheal tube is also needed if a patient is unconscious or has a brain injury. A less common reason for intubating a patient is to keep the airway open. This is seen most commonly in patients with tumors or growths in the neck or upper chest.

The usual route for inserting an endotracheal tube is through the mouth. This is called an oral endotracheal tube. Less frequently, the endotracheal tube is inserted through the nose. This is called a nasal endotracheal tube. After intubation, the cuff is inflated with air. As shown in the image below, this is done by attaching a syringe to the pilot balloon. The pilot balloon is connected to the cuff by a thin tube. As the syringe supplies pressurized air, the pilot balloon and cuff inflate

A soft, usually donut shaped balloon is located around the outside of the distal end of the endotracheal tube. This is called the “cuff”. After intubation, the cuff is inflated with air. This is typically done by attaching a syringe to a pilot balloon. The pilot balloon is connected to the cuff by a thin tube often called a pilot tube. As the syringe supplies pressurized air via the pilot tube, the pilot balloon and the cuff inflate. The inflated cuff serves two purposes. First, it reduces the number of oral secretions that can travel down the outside of the tube and into the lung. This is important because inserting an endotracheal tube into the airway will “wedge” the epiglottis into an open position. Although the cuff reduces the amount of secretions that can enter the lung, it does not completely prevent it. For this reason, patients on ventilators are at risk for developing pneumonia from secretions.

The second reason for the cuff is to keep any mechanical “breaths” from leaking out of the lung around the tube. When a patient is given a breath with the ventilator, it is desired that the breath to go in and out through the tube. Without a cuff, the breath would go in the tube but part of it would escape around the cuff before it was able to reach the lung. As long as the patient has a cuffed endotracheal tube in place for purposes of ventilation, the cuff will need to be inflated.

Under current practice, the endotracheal tube (“ETT”) cuff (See FIG. 1) is inflated once the ETT is inserted into the patient's trachea using a number of devices. Once the ETT is optimally placed, most practitioners use a syringe filled with air, attach it to the one-way valve in the pilot balloon and infuse air through the pilot balloon line to inflate the cuff. The inflated ETT Cuff prevents “blow-by” of ventilator (or anesthesia equipment) supplied gases to the patient's lungs. Without this seal in the patient's trachea, there would be little possibility to apply positive pressure to the patient's lungs from one of these machines or via a manual resuscitator.

Many investigations have been conducted as to the correct amount of inflation pressure one should use with ETT Cuffs. Generally speaking, ETT cuff inflation pressures are thought to be ideal in the 20 to 30 cmH2O range. Too little pressure allows for some “blow-by”, but a more insidious result is the possibility that contaminated sub-glottic fluids have the opportunity to leak past the flaccid cuff into the patient's lungs where pneumonia may fester (“micro-aspiration”).

On the other hand, an ETT cuff inflated at too high a pressure is a risk for damage to the patient's tracheal mucosa. Tracheal mucosa damage ranges from minor irritation and sore throats to severe voice and tracheal damage and possible ulceration and death. There are many studies showing that practitioners have a propensity to over-inflate cuffs up to pressures of 100 cmH2O and higher.

Presently, many respiratory practitioners either use the generally accepted practices of minimal occluding volume (MOV) or minimal leak technique (MLT) when they inflate the ETT

Cuff. Other practitioners do not use either one of these techniques rather they just “guess” that the ETT Cuff is inflated to the correct pressure.

In clinical practice, it is well known that humidity can and will build up in ventilatory circuits due to heated inspiratory gas being exposed to room temperature while being delivered to the patient. This differential in temperature between room air and delivered gas causes “rainout”, or water droplets in the patient circuit (the tubing which is attached to the patient on one end, and the ventilator on the other). This phenomenon also exists to a lesser degree in the pilot balloon valve.

What is less known is that humidity will also build up in the pilot balloon line as a result of water vapor passing across the ET cuff itself. This is particularly true with newer generation cuffs constructed from polyurethane “PU” (vs. traditional cuffs which are constructed of poly vinyl chloride “PVC”). PU cuffs have become more and more popular in clinical use, due their documented effect of lowering ventilator-associated pneumonia rates. Covidien, Halyard Health, Parker Medical (Salter Labs) and several other manufacturers produce PU cuffs.

These PU cuffs create a superior tracheal seal and are designed to reduce the leakage of potentially infectious secretions into the lungs. While exhibiting superior strength and tracheal sealing qualities, the micro-thin polyurethane material used to make the endotracheal tube cuff may exhibit water condensation within the cuff. This condensation is formed primarily by two factors.

The first factor is that the material properties of polyurethane in combination with the micro-thin design allow the cuff to be more permeable, or breathable, than other conventional polyvinyl chloride (PVC) cuffs. Humidified air in contact with the cuff allows water molecules to physically/chemically migrate into the cuff. In other words, the air inside the cuff becomes humidified.

The second factor is that the temperature difference between the air inside the cuff 4, FIG. 1 (T1) and the ventilation gas 6 (T2), flowing through the main tube shaft 8 causes the humidified air 5 inside the cuff 4 to condense 9 over time. The greater the temperature difference (T1 greater than T2), the more likely condensation 9 will appear. This has been confirmed through lab testing. Numerous factors impact the amount of humidity 9 that will collect (condense) in the cuff 4 and cuff inflation or pilot line 7, including dew point, patient temperature, use of instilled saline prior to suctioning, heated and/or humidified air circuits, room temperature, etc.

The transfer of moisture 9 into the cuff 4 is therefore on the molecular level. Thus, although the cuffs are breathable, they are not permeable to microorganisms. According to polymer material experts, permeability is driven by vapor pressure across the membrane which is impacted by temperature only, vs. airway or cuff pressure. Conventional PVC cuffs are not as permeable as PU cuffs, but they also can exhibit some condensation, although less than PU cuffs.

Humidity/condensation 9 not only builds up in the cuff 4, but also in the pilot balloon line 7 and the pilot balloon (not shown but well known) itself. When water droplets of sufficient size form to completely obstruct the pilot line inner lumen, it becomes impossible to accurately measure cuff pressures. In order to measure cuff pressure, the measurement device must have an unobstructed column of air straight through to the cuff itself. If there are water droplets obstructing this path, the measurement device will only be able to measure the pressure between the device itself and the nearest obstructing droplet.

If cuff pressures cannot be reliably ascertained due to this condensation, intubated patients remain at risk for micro-aspiration and tracheal damage, despite efforts to monitor cuff pressures. Furthermore, if these droplets occur out of sight—in the portion of the ETT pilot line that is deep inside the patient, for example—the clinician will have no idea there is an obstruction and will assume cuff pressures are being adequately maintained, when in fact, they are not.

Accordingly, what is needed is a device that takes the “guessing” out of the initial inflation, moreover, it continues to monitor the ETT Cuff pressure and adjusts automatically the ETT Cuff pressure to the target pressure value (“set point”) that the respiratory practitioner programs on the device.

### SUMMARY OF THE INVENTION

The invention features a device for purging condensation/moisture from an air line coupled to an air cuff on an artificial airway. The device comprises a pressure measurement device, coupled to an air line which in turn is coupled to an air cuff. The pressure measurement device is configured for monitoring and measuring the air pressure in the air line and coupled air cuff, and for providing a signal indicating the measured air pressure.

An air pressure control device is coupled to the air line which in turn is coupled to the air cuff. The air pressure control device is configured for increasing or decreasing air pressure in the air line and coupled air cuff in response to an air pressure control device control signal. A pressure regulator is provided and configured for receiving the signal indicating the measured air pressure from the pressure measurement device and responsive to a preprogrammed air pressure value, for determining whether the measured air pressure is equal to, greater than or less than the preprogrammed air pressure value. The pressure regulator is coupled to the air pressure control device and configured for providing the air pressure control device control signal. The pressure regulator is further configured to be responsive to a determination that the measured air pressure is greater than the preprogrammed air pressure value, for providing a first air pressure control device control signal configured for causing the air pressure control device to decrease the air pressure in the air line and coupled air cuff, and configured to be responsive to a determination that the measured air pressure is less than the preprogrammed air pressure value, for providing a second air pressure control device control signal configured for causing the air pressure device to increase the air pressure in the air line and coupled air cuff.

An air line purge regulator device is provided which is responsive to one or more air line purge control values and coupled to the air pressure control device, and configured for providing a first air pressure control device purge control signal for causing the air pressure control device to increase the air pressure in the air line and coupled air cuff to a predetermined purge air pressure value for a predetermined period of time at predetermined intervals of time and also configured for providing a second air pressure control device purge control signal for causing the air pressure control device to decrease the air pressure in the air line and coupled air cuff to the preprogrammed air pressure value.

In one embodiment, the preprogrammed air pressure value and air line purge control values are provided by user input and may be stored in a device processor/controller. In the preferred embodiment, the device processor/controller is configured and programmed to function as both the a pressure regulator and the an air line purge regulator device and provides the both the preprogrammed air pressure value to the pressure regulator and the first and second air pressure control device purge control signals to the air line purge regulator.

The device processor/controller may be responsive to the signal indicating the measured air pressure from the pressure measurement device, for providing at least one of a visual display and an audible display if the measured air pressure exceeds or is less than the preprogrammed air pressure value.

In a preferred embodiment, the device processor/controller is a microprocessor based or programmable logic array type of device that comprises both a pressure regulator system as well as an air line purge regulator device. The pressure regulator system is configured for receiving the signal indicating the measured air pressure from the pressure measurement device and responsive to a preprogrammed air pressure value, for determining whether the measured air pressure is equal to, greater than or less than the preprogrammed air pressure value, the pressure regulator system coupled to the air pressure control device and configured for providing the air pressure control device control signal, the pressure regulator system further configured to be responsive to a determination that the measured air pressure is greater than the preprogrammed air pressure value, for providing a first air pressure control device control signal configured for causing the air pressure control device to decrease the air pressure in the air line and coupled air cuff, and configured to be responsive to a determination that the measured air pressure is less than the preprogrammed air pressure value, for providing a second air pressure control device control signal configured for causing the air pressure device to increase the air pressure in the air line and coupled air cuff,

The air line purge regulator device is responsive to one or more air line purge control values and coupled to the air pressure control device, and configured for providing a first air pressure control device purge control signal for causing the air pressure control device to increase the air pressure in the air line and coupled air cuff to a predetermined purge air pressure value for a predetermined period of time at predetermined intervals of time, the air line purge regulator device further configured for providing a second air pressure control device purge control signal for causing the air pressure control device to decrease the air pressure in the air line and coupled air cuff to the preprogrammed air pressure value.

### BRIEF DESCRIPTION OF THE DRAWINGS

These and other features and advantages of the present invention will be better understood by reading the following detailed description, taken together with the drawings wherein:

FIG. 1 is a schematic representation of an air line, attached to an air cuff or other inflation apparatus;

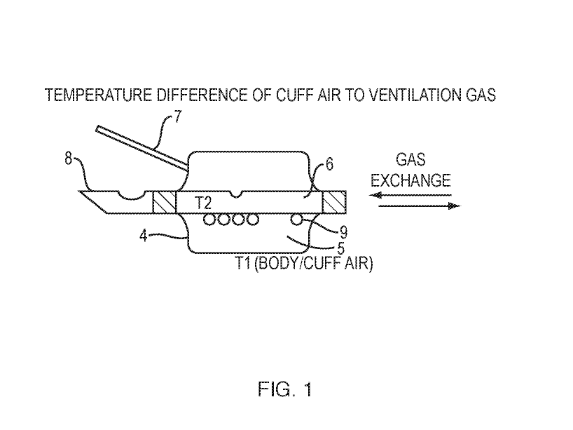

FIG. 2 is a block diagram of a device for purging an air line, attached to an air cuff or other inflation apparatus of accumulated moisture in accordance with the present invention; and

FIG. 3 is a block diagram of the air or pneumatic circuit of the device according to the present invention.

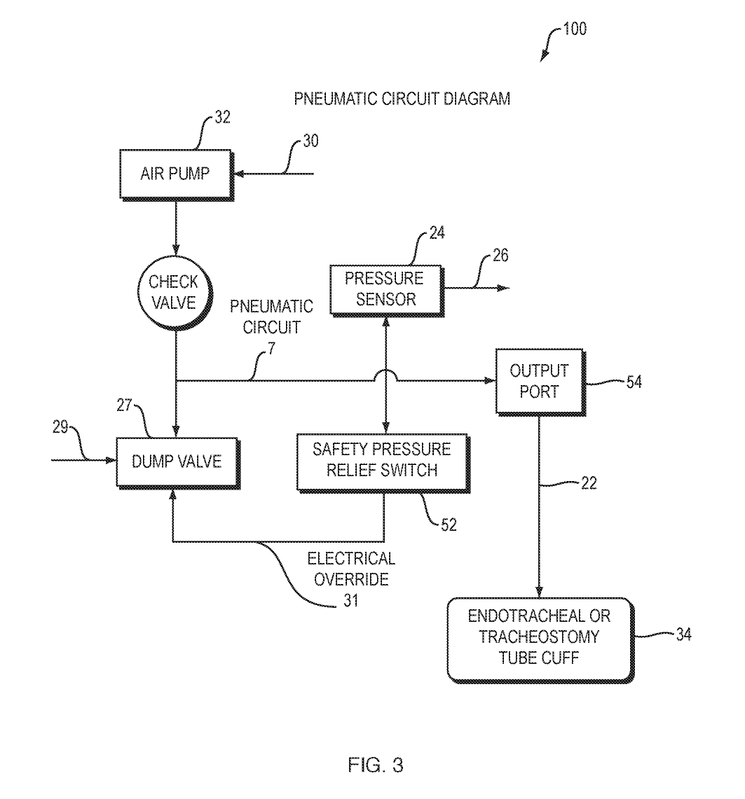

### DETAILED DESCRIPTION OF THE PREFERRED EMBODIMENTS

The present invention features a device and method that provides an automatic and/or manual “purge” cycle in an air cuff line (pilot line) on an artificial airway to prophylactically rid the pilot balloon valve and line of any condensate, thereby keeping the communication between the air-filled cuff and the cuff pressure monitor constant. Examples of these airways would include endotracheal tubes, tracheostomy tubes, and supra-glottic airways

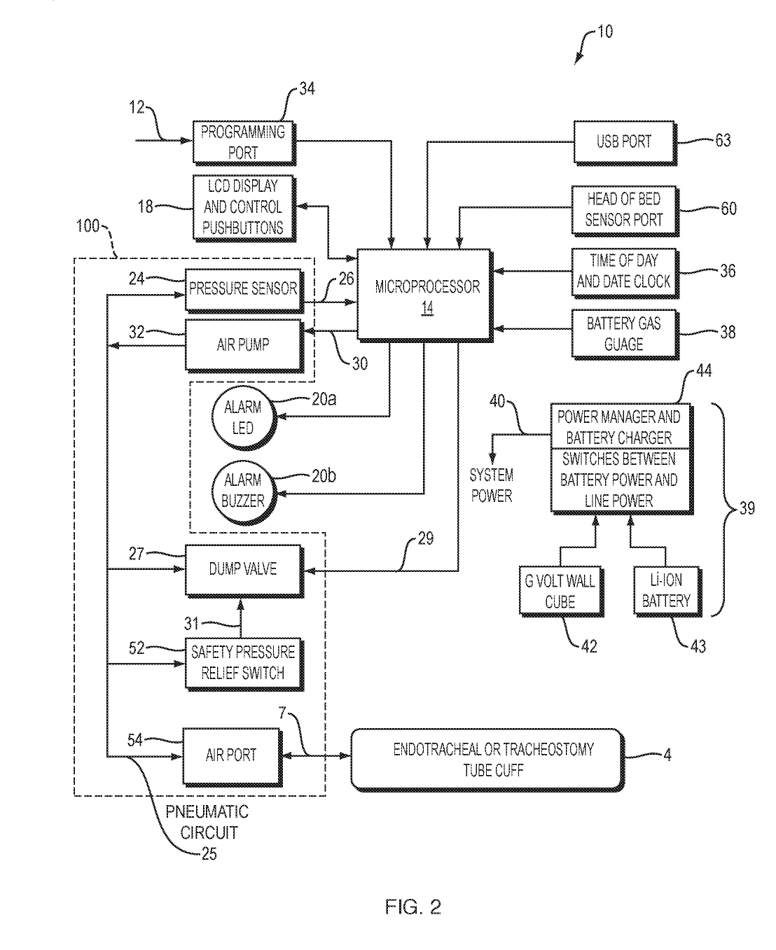

As shown schematically in the block diagram of FIG. 2, the device 10 of the present invention is configured either as a small standalone enclosure inside of which resides all of the hardware and software to effectuate the functionality described herein or alternatively, includes the required hardware, software and pneumatic circuitry built into a larger device. A processing device 14, such as a microprocessor or programmable logic array, is responsive to appropriate pre-programmed software instructions stored in memory (not shown but well known) to coordinate and control the operation of the device 10. Input 12 from a user in the form of keypad or other type of well-known input device input may be provided to the software processor 14 via a programming port 34. The software processor 14 controls an on board or external display 18 and/or audible or visual alarms 20. The device 10 is coupled to a cuff 4 by means of air line (pilot line) 7 coupled to the air port 54 of the device 10.

In operation, the air line 7 is coupled to a pressure measurement device 24 such as a transducer or other pressure type sensor, which measures the air pressure in the cuff 4, by means of the air port 54 coupled via a pneumatic circuit 25, and provides a signal 26 indicative of the air pressure to the microprocessor 14. The microprocessor 14 utilizes the pressure sensor signal 26 to measure the air pressure in the air line 7 and cuff 4 to determine whether the pressure is appropriate, too high or too low based on the user input 12 and/or preprogrammed values in the software process of the microprocessor 14. If the air pressure in the air line 7 and cuff 4 is as programmed, no further action is required. However, if the air pressure is too high, the microprocessor 14 provides a signal 29 to dump valve 27 which causes the air pressure device 32 to vent air from the air line 7 and cuff to ambient air. If the air pressure in air line 7 and cuff as measured by the pressure measurement device 24 is to low, the microprocessor 14 provides signal 30 to the air pump or other air pressure device 32, which causes the air pressure device 32 to increase the pressure in the air line 7 and cuff 4 providing increased air pressure in the cuff 4. The combination of the pressure measurement device/sensor 24, the air pump 32, dump valve 27 and microprocessor 14 cooperate together to provide a real time constant feedback loop and adjustment of the air pressure in the cuff 4.

One aspect of the present invention is a feature of automatically purging any moisture out of air line 7 into the cuff 4 such that accurate air pressure measurements are made. According to one aspect of the present invention, purge line control and regulation is controlled by the software controlled microprocessor or programmable logic array 14 which may include preprogrammed instructions or user programmable instructions input by means of user input 12 on programming port 34 to control this feature. At predetermined time intervals, the microprocessor 14 causes the air pressure pump/device 32 to impart a predetermined amount of pressure on air line 7 for a predetermined period of time in order to purge any moisture from within the air line 7. Any accumulated moisture in the air line 7 is pushed into the cuff 4. The appropriate pressure in the cuff 4 is maintained utilizing the pressure measurement sensor 24 and dump valve 27 to make sure that the appropriate amount of pressure for the appropriate amount of time is maintained on the air line 7. Once the air pressure has been maintained for the predetermined period of time, microprocessor 14 activates the dump valve 27 to release the increased pressure from air line 7 and cuff 4 thereafter maintaining the standard desired air pressure.

The invention is, in one embodiment, a stand-alone monitor that is preferably equipped with an integrated USB 63 port and alarms 20, as well as a power system 39 including a rechargeable battery backup 43, AC wall power source 42, and power manager and battery charger 44 all working together to provide system power 40. The monitor of the invention is used in conjunction with a disposable, single-patient use tubing set, which is connected to the artificial airway at the pilot balloon. In another embodiment, the present invention can be integrated into another device such as a ventilator or anesthesia machine. Additional inputs such as a head of bed position sensor 60, time of day and date clock 36, and battery gas gauge 38 may be provided.

To prevent the unwanted effects of undesirable cuff pressures, the invention checks the cuff pressure every millisecond; and any time its pressure sensor 24 notes a drop in pressure of more than 2 cmH2O from the set point, it will immediately entrain room air into the cuff 4 to bring it to within +/−2cmH2O of the set point. In the event that the set point cannot be achieved within 30 seconds (i.e.; a massive leak) an alarm will sound.

If the transducer sensor 24 notes an increase in pressure of more than 2 cm H2O around the set point that persists for at least 45 seconds, it will vent to room air to bring the cuff pressure back to +/−2 cmH2O by activating dump valve 27. If a high pressure condition cannot be resolved within 2 minutes, an alarm will sound to notify the clinician.

The invention also includes an automatic line “purge” cycle to prophylactically rid the pilot balloon, valve and line 7 of any condensate, thereby keeping the communication between the air-filled cuff 4 and the cuff pressure monitor 10 constant. For example, the preferred embodiment contemplates that every 30 minutes, the device according to the present invention will deliver 85 cm H2O for 25 seconds, which will “push” any humidification or moisture from the pilot line 7 line into the cuff 4, where it will not interfere with cuff pressure readings. 85 cm H2O is believed to be the lowest pressure that will effectively clear the pilot line 7 in a short time period however higher or lower purge pressures and longer or shorter times are potentially contemplated and within the scope of the present invention. In the unlikely event that this automatic purge is unsuccessful, the clinician can also press the “Inflate” key to trigger a manual “purge” cycle or may otherwise initiate a manual purge cycle.

The present invention will automatically bring the cuff pressure back to +/−2 cm H2O around the set point after the 25 seconds “purge” cycle; essentially immediately after the “purge” cycle completes. It is as yet undetermined whether or not this frequency of purge cycle will be adequate to keep the line free of condensate to ensure accurate cuff pressure readings. The belief is that every half hour should be sufficient, but actual clinical use will provide useful data in this regard.

Accordingly, the present invention provides a system and method for maintaining air pressure within the air cuff on an artificial airway while also providing a system and method for purging the air line between the controller and the artificial airway cuff to assure accurate readings of air pressure within the cuff. Modifications and substitutions by one of ordinary skill in the art are considered to be within the scope of the present invention, which is not to be limited except by the allowed claims and their legal equivalents.

What is claimed is:

1. A device for purging condensation/moisture from an air line coupled to an air cuff on an artificial airway, the device comprising:
a pressure measurement device, coupled to an air line which in turn is coupled to an air cuff, said pressure measurement device configured for monitoring and measuring the air pressure in said air line and coupled air cuff, and for providing a signal indicating said measured air pressure;
an air pressure control device, coupled to said air line which in turn is coupled to said air cuff, said air pressure control device configured for increasing or decreasing air pressure in said air line and coupled air cuff in response to an air pressure control device control signal;
a pressure regulator, configured for receiving said signal indicating said measured air pressure from said pressure measurement device and responsive to a preprogrammed air pressure value, for determining whether said measured air pressure is equal to, greater than or less than said preprogrammed air pressure value, said pressure regulator coupled to said air pressure control device and configured for providing said air pressure control device control signal, said pressure regulator further configured to be responsive to a determination that said measured air pressure is greater than said preprogrammed air pressure value, for providing a first air pressure control device control signal configured for causing said air pressure control device to decrease said air pressure in said air line and coupled air cuff, and configured to be responsive to a determination that said measured air pressure is less than said preprogrammed air pressure value, for providing a second air pressure control device control signal configured for causing said air pressure device to increase said air pressure in said air line and coupled air cuff; and
an air line purge regulator device, responsive to one or more air line purge control values and coupled to said air pressure control device, and configured for providing a first air pressure control device purge control signal for causing said air pressure control device to increase said air pressure in said air line and coupled air cuff to a predetermined purge air pressure value for a predetermined period of time at predetermined intervals of time, said air line purge regulator device further configured for providing a second air pressure control device purge control signal for causing said air pressure control device to decrease said air pressure in said air line and coupled air cuff to said preprogrammed air pressure value.

2. The device of claim 1, wherein said preprogrammed air pressure value and air line purge control values are provided by user input.

3. The device of claim 1, wherein said preprogrammed air pressure value and said air line purge control values are stored in a device processor/controller.

4. The device of claim 3, wherein said device processor/controller is configured and programmed to function as both said a pressure regulator and said an air line purge regulator device.

5. The device of claim 4, wherein said device processor/controller provides said preprogrammed air pressure value to said pressure regulator and said first and second air pressure control device purge control signals to say air line purge regulator.

6. The device of claim 3, wherein said device processor/controller is responsive to said signal indicating said measured air pressure from said pressure measurement device, for providing at least one of a visual display and an audible display if said measured air pressure exceeds or is less than said preprogrammed air pressure value.

7. A device for purging condensation/moisture from an air line coupled to an air cuff on an artificial airway, the device comprising:
a pressure measurement device, coupled to an air line which in turn is coupled to an air cuff, said pressure measurement device configured for monitoring and measuring the air pressure in said air line and coupled air cuff, and for providing a signal indicating said measured air pressure;
an air pressure control device, coupled to said air line which in turn is coupled to said air cuff, said air pressure control device configured for increasing or decreasing air pressure in said air line and coupled air cuff in response to an air pressure control device control signal; and
a device processor/controller, said device processor/controller comprising:
a pressure regulator system, configured for receiving said signal indicating said measured air pressure from said pressure measurement device and responsive to a preprogrammed air pressure value, for determining whether said measured air pressure is equal to, greater than or less than said preprogrammed air pressure value, said pressure regulator system coupled to said air pressure control device and configured for providing said air pressure control device control signal, said pressure regulator system further configured to be responsive to a determination that said measured air pressure is greater than said preprogrammed air pressure value, for providing a first air pressure control device control signal configured for causing said air pressure control device to decrease said air pressure in said air line and coupled air cuff, and configured to be responsive to a determination that said measured air pressure is less than said preprogrammed air pressure value, for providing a second air pressure control device control signal configured for causing said air pressure device to increase said air pressure in said air line and coupled air cuff; and
an air line purge regulator device, responsive to one or more air line purge control values and coupled to said air pressure control device, and configured for providing a first air pressure control device purge control signal for causing said air pressure control device to increase said air pressure in said air line and coupled air cuff to a predetermined purge air pressure value for a predetermined period of time at predetermined intervals of time, said air line purge regulator device further configured for providing a second air pressure control device purge control signal for causing said air pressure control device to decrease said air pressure in said air line and coupled air cuff to said preprogrammed air pressure value.
